(19) United States
(12) Patent Application Publication
(10) Pub. No.: US 2013/0316417 A1
(43) Pub. Date: Nov. 28, 2013

(54) PROCESS FOR THE CONVERSION OF GLYCEROL TO 1,3-PROPANEDIOL

(71) Applicants: Martin J.T. Reaney, Saskatoon (CA); Monique Haakensen, Saskatoon (CA); Darren Korber, Saskatoon (CA); Takuji Tanaka, Saskatoon (CA); Kornsulee Ratanapariyanuch, Saskatoon (CA)

(72) Inventors: Martin J.T. Reaney, Saskatoon (CA); Monique Haakensen, Saskatoon (CA); Darren Korber, Saskatoon (CA); Takuji Tanaka, Saskatoon (CA); Kornsulee Ratanapariyanuch, Saskatoon (CA)

(73) Assignee: University of Saskatchewan, Saskatoon, SK (CA)

(21) Appl. No.: 13/877,973

(22) PCT Filed: Oct. 7, 2011
(86) PCT No.: PCT/CA2011/050633

§ 371 Date: Aug. 13, 2013

Related U.S. Application Data
(60) Provisional application No. 61/391,113, filed on Oct. 8, 2010.

(51) Int. Cl. C12P 7/18

(52) U.S. Cl. CPC C12P 7/18
USPC 435/158; 435/252.9

(57) ABSTRACT

The present application includes a process and a microorganism of the genus Lactobacillus that converts glycerol to 1,3-propanediol. The conversion is accomplished by a proprietary microorganism that is easily cultured in glycerol rich waste products of ethanol production, such as thin stillage.

### RELATED APPLICATIONS

The present application claims the benefit of priority of co-pending U.S. provisional patent application No. 61/391,113 filed on Oct. 8, 2010, the contents of which are incorporated herein by reference in their entirety.

### FIELD OF THE APPLICATION

The present application is directed to processes for the conversion of glycerol to 1,3-propanediol, in particular using one or more microorganisms isolated from thin stillage.

### BACKGROUND OF THE APPLICATION

One of the concerns related to biofuel production is the management of by-products of the processes. In bioethanol production, yeasts metabolize sugar feedstock into ethanol and glycerol. The amount of glycerol reaches 5˜8% of ethanol. In the case of biodiesel, triglycerides in plant seed oils are hydrolyzed during processing; one triglyceride molecule is hydrolyzed into three fatty acids, which are easily converted to diesel oils, and one equivalent of glycerol. Typically this process yields glycerol and biodiesel in the ratio of one to ten by weight. If biofuel production increases as planned, nearly 140,000 tonnes of glycerol will be available from biofuel production in Canada alone by 2010. This is a huge supply of glycerol, considering the global market of glycerol was 800,000 tonnes in 2005. The large increase in production of these products has and will continue to produce a glut of glycerol in the marketplace and result in a lowered price for this commodity.

The inevitable formation of glycerol during biofuel production has driven the demand to develop methods for glycerol utilization. Glycerol carbon, however, is in a highly oxidized state, and thus has low reactivity. It is challenging to produce value-added products from glycerol. The largest application of glycerol is as a stabilizer found in drug/pharmaceutical, cosmetics/personal care products, and foods. In these applications, glycerol is used without chemical conversion. The demands for these purposes are not very high compared to the expected expansion of biofuel production. This disproportion of supply and demand of glycerol will produce an excess amount of glycerol in the near future driving glycerol prices down. Excess glycerol that cannot find further application will be discarded. This means that part of the energy input in the biofuel production process is wasted along with part of the starting materials. The costs of the wasted energy and glycerol disposal further pose unrecoverable expenditures on the balance sheets of biofuel production, as well as possible environmental impacts. Considering these factors, it is desirable to develop methods to convert glycerol into other more chemically active compounds (i.e., platform chemicals). To date, many studies have been conducted to convert glycerol into platform chemicals such as 3-ketomaloic acid, 1,3-dihydroxyacetone, and acrolein, using the approaches developed in petrochemical industries, i.e., using chemical catalysts. However, the proposed methods fail to satisfy the requirements of industrial applications. For example, an acidic catalyst can dehydrate glycerol into acrolein at 250 to 340° C. in the presence of water. The high cost associated with this conversion makes it not commercially feasible. Many glycerol conversion processes have similar issues limiting their use in industrial situations. While there are difficulties in the conversion, some glycerol derivatives can be very good platform chemicals. Acrolein (didehydrated glycerol) can be converted into acrylic acid, which is a building material of many polymers. 1,3-propanediol (1,3-PD or monodehydroxy glycerol) is processed into polyester fibre.

### SUMMARY OF THE APPLICATION

Bacteria have been isolated from ethanol thin stillage that are capable of converting glycerol to 1,3-propanediol (1,3-PD). These bacteria also convert lactic acid to acetic acid. It is proposed that the industrial application of these organisms will provide a direct route to the commercial production of high-value chemicals directly linked to the production of ethanol. This option would be desirable because bioprocessing thin stillage and/or distiller's solubles in this manner would have low associated costs. 1,3-PD is formed from glycerol through a two-step enzymatic process. First, glycerol dehydratase (GD) produces 3-hydroxypropionaldehyde (3-HPA) by dehydrating glycerol. The 3-HPA is then converted to 1,3-PD by the 1,3-propanediol oxidoreductase enzyme (DhaT) (see Scheme 1).

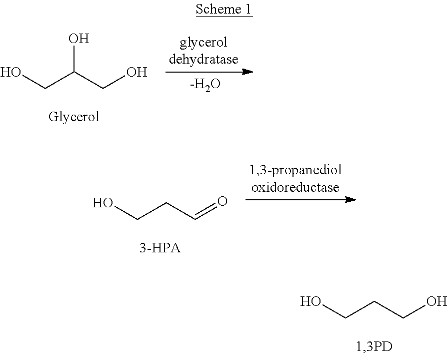 [SMILES: OCC(O)CO ; "-H2O" ; O=CCCO ; OCCCO]

Both enzymes have been found in several bacteria, including the genera Citrobacter, Clostridium, Klebsiella, and Lactobacillus. Of genera that have been previously reported to possess the ability to convert glycerol to 1,3-PD, only Lactobacillus possess generally regarded as safe (GRAS) status.

The present application includes a microorganism cultured from ethanol thin stillage that converts glycerol, and optionally lactic acid, into commercially useful products. In an embodiment, the commercially useful product obtained from glycerol is 1,3-PD and the commercially useful product obtained from lactic acid is acetic acid.

In an embodiment, the microorganism belongs to the genus Lactobacillus. In a specific embodiment, the microorganism is Lactobacillus panis PM1A, Lactobacillus panis PM1B or Lactobacillus buchneri PM3. In a further specific embodiment, the microorganism is Lactobacillus panis PM1 deposited under accession number 180310-01 at the International Depository Authority of Canada located in, National Microbiology Laboratory, Public Health Agency of Canada, 1015 Arlington Street, Winnipeg, Manitoba, R3E 3R2, Canada on Mar. 18, 2010. In a further specific embodiment, the microorganism is Lactobacillus buchneri PM3 deposited under accession number 280910-01 at the International Depository Authority of Canada located in, National Microbiology Laboratory, Public Health Agency of Canada, 1015 Arlington Street, Winnipeg, Manitoba, R3E 3R2, Canada on Sep. 28, 2010.

The present application also includes a process for the production of 1,3-propanediol (1,3-PD) comprising contacting a microorganism cultured from ethanol thin stillage with glycerol under conditions for the formation of 1,3-PD and, optionally, isolating the 1,3-PD. In a specific embodiment of the application the microorganism is Lactobacillus panis PM1A, Lactobacillus panis PM1B or Lactobacillus buchneri PM3. In a further specific embodiment, the microorganism is Lactobacillus panis PM1 deposited under accession number 180310-01 at the International Depository Authority of Canada located in, National Microbiology. Laboratory, Public Health Agency of Canada, 1015 Arlington Street, Winnipeg, Manitoba, R3E 3R2, Canada on Mar. 18, 2010. In a further specific embodiment, the microorganism is Lactobacillus buchneri PM3 deposited under accession number 280910-01 at the International Depository Authority of Canada located in, National Microbiology Laboratory, Public Health Agency of Canada, 1015 Arlington Street, Winnipeg, Manitoba, R3E 3R2, Canada on Sep. 28, 2010.

The present application also includes a process for the conversion of lactic acid to acetic acid comprising contacting a microorganism cultured from ethanol thin stillage with lactic acid under conditions for the formation of acetic acid and, optionally, isolating the acetic acid. In an embodiment the conversion of lactic acid to acetic acid is performed at the same time as the conversion of glycerol to 1,3-PD in a co-fermentation process.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The application will now be described in greater detail with reference to the drawings in which:

FIG. 1 shows the DNA sequence for the 16S rRNA gene from PM1A and PM1B [SEQ ID NO:1].

FIG. 2 shows the DNA sequence for the 16S-23S rRNA internal transcribed spacer gene from PM1A and PM1B [SEQ ID NO:2].

FIG. 3 shows the DNA sequence for the cpn60 gene from PM1A and PM1B [SEQ ID NO:3].

FIG. 4 shows the DNA sequence for the glycerol dehydratase gene, large subunit [SEQ ID NO:4], medium subunit [SEQ ID NO:5] and small subunit [SEQ ID NO:6].

FIG. 5 shows the DNA sequence for the cobyric acid gene from PM1A and PM1B [SEQ ID NO:7].

FIG. 6 shows the DNA sequence for the 16S rRNA gene from PM3 [SEQ ID NO:8].

FIG. 7 is a ¹H NMR spectrum of thin stillage pre-conversion with a microorganism of the present application.

FIG. 8 is a ¹H NMR spectrum of thin stillage post-conversion with a microorganism of the present application.

FIG. 9 is a ¹H NMR spectrum of distillers solubles pre-conversion with a microorganism of the present application.

FIG. 10 is a ¹H NMR spectrum of distillers solubles post-conversion with a microorganism of the present application.

### DETAILED DESCRIPTION

### Definitions

The following definitions, unless otherwise stated, apply to all aspects and embodiments of the present application.

The term “thin stillage” as used herein refers to a complex aqueous solution comprising ions, organic compounds and other compounds, which is obtained from the fuel ethanol industry as a common waste by-product. Thin stillage is produced from the fermentation, by yeast and/or other microorganisms, of starches and sugar, which results in an ethanol containing slurry called a beer. After distillation of the beer to remove the ethanol, a slurry called stillage remains. Filtering the solids from the stillage, provides the thin stillage. Thin stillage may also be thickened through evaporation to a product called distiller's solubles. As used herein, the term “thin stillage” also includes distiller's solubles.

The term “commercially useful products” as used herein refers to compounds that are sold commercially or can be used to make compounds that are sold commercially.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Microorganisms of the Application

The present application includes a microorganism cultured from ethanol thin stillage that converts glycerol, or optionally lactic acid, into commercially useful products. In an embodiment, the microorganism belongs to the genus Lactobacillus. In a specific embodiment, the microorganism is Lactobacillus panis strain PM1A, PM1B or Lactobacillus buchneri strain PM3. Lactobacillus panis strain PM1A and PM1B are genetically identical however have different morphologies. PM1A is smooth, semitransparent to white to creamy in colour, circular in form, entire margin, flat or convex elevation and PM1B is dry in appearance, undulate, raised, rough, and adhers to agar.

In a further specific embodiment, the microorganism is Lactobacillus panis PM1 deposited under accession number 180310-01 at the International Depository Authority of Canada located in, National Microbiology Laboratory, Public Health Agency of Canada, 1015 Arlington Street, Winnipeg, Manitoba, R3E 3R2, Canada on Mar. 18, 2010. In a further specific embodiment, the microorganism is Lactobacillus buchneri PM3 deposited under accession number 280910-01 at the International Depository Authority of Canada located in, National Microbiology Laboratory, Public Health Agency of Canada, 1015 Arlington Street, Winnipeg, Manitoba, R3E 3R2, Canada on Sep. 28, 2010. Lactobacillus panis PM1A and Lactobacillus panis PM1B genetically identical, however their appearance is different and they aggregate differently.

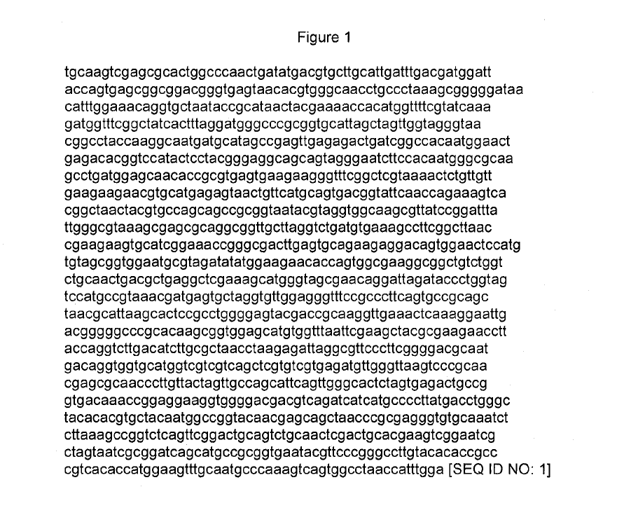

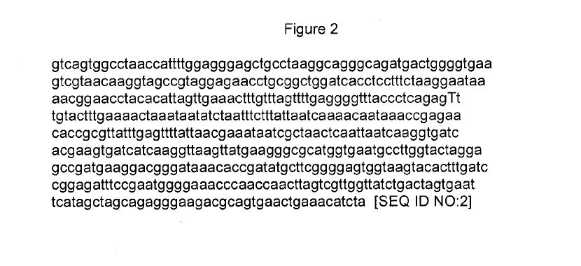

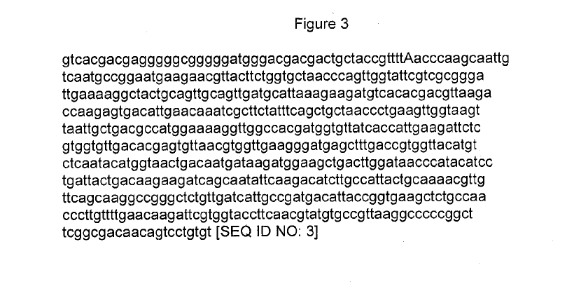

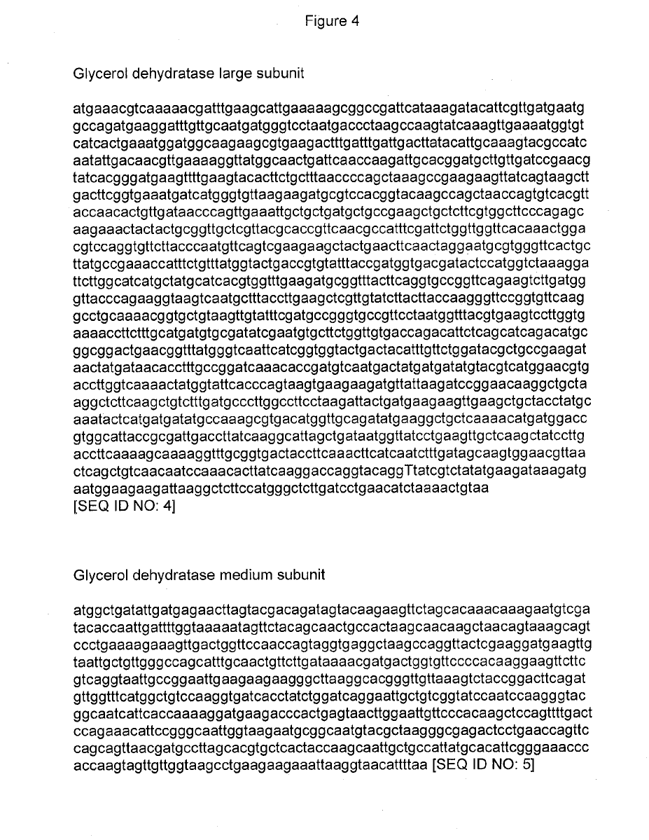

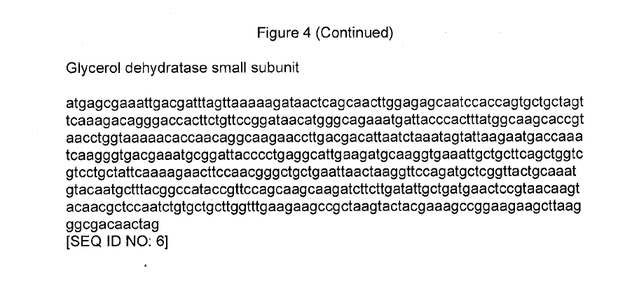

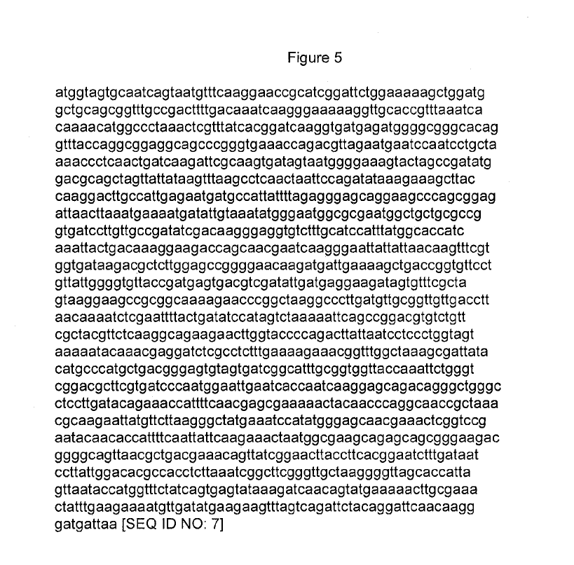

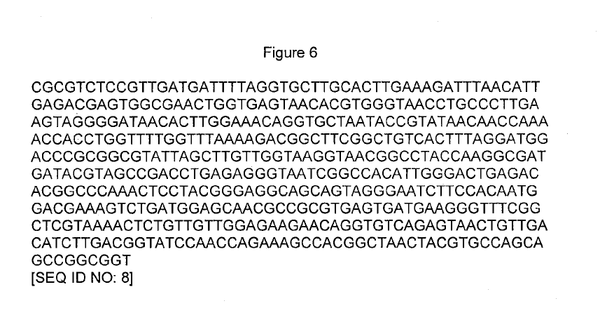

The Lactobacillus microorganisms cultured from thin stillage that has been shown convert glycerol to 1,3-PD is further characterized as follows:
lives aerobically and/or anaerobically;
converts glycerol and, optionally lactic acid, to a commercially useful product under aerobic and/or anaerobic conditions;
conversion of glycerol to commercially useful products is not inhibited by high concentrations of the end-product;
is adapted to live at temperatures above 45° C. under acidic conditions;
lives and converts glycerol to commercially useful products at pH lower than 3.8;
can be cultured in de Mann Rogosa Sharpe (MRS) broth or agar (pH 6.5, 5.7, or 4.2, depending on manufacturer);
can be cultured in broth or agar made by autoclaving stillage;
can be cultured in broth or agar made by autoclaving stillage and mixing with MRS medium in any ratio;
can grow in and convert glycerol to 1,3-PD, and optionally lactic acid to acetic acid, in distiller's solubles diluted less than 50% with water;
can grow in and convert thin stillage from different origins;
can be stored at 4° C. for >1 year in any of the above described growth mediums and remain viable and capable of performing the above described conversion of glycerol to 1,3-PD, and optionally lactic acid to acetic acid;
appears as smooth, semitransparent to white to creamy, circular form, entire margin, flat or convex elevation (PM1A), but also as dry in appearance, undulate, raised, rough, and adhering to agar (PM1B). The bacterial strain can switch between four morphology types;
Acidogenic characteristics identified by adding pH indicator to growth medium agar and by testing liquid medium with a pH probe;
Gram-stain and appearance under microscope characteristic of Lactobacillus;
Tolerant of living with bacteria of the Acetobacter and/or Bacillus genera and other species of the genus Lactobacillus;
Tolerant of living with consortia of wild bacteria, yeasts, and fungi;
Tolerant of living in thin stillage which had been treated with Virginiamycin during fermentation;
Tolerant of living in thin stillage from different origins, and the bacteria, yeasts, fungi, and their by-products within that stillage;
Possesses at least one, and suitably all, of the following DNA sequences: Lactobacillus panis PM1A and PM1B: 16S rRNA gene sequence as shown in FIG. 1 [SEQ ID NO:1]; 16S-23S rRNA internal transcribed spacer (ITS) gene sequence as shown in FIG. 2 [SEQ ID NO:2]; cpn60 gene sequence as shown in FIG. 3, [SEQ ID NO:3]; glycerol dehydratase gene sequence, as shown in FIG. 4 [SEQ ID NO:4], [SEQ ID NO:5] and [SEQ ID NO:6]; and cobyric acid synthetase gene sequence as shown in FIG. 5 [SEQ ID NO:7]. Lactobacillus buchneri PM3: 16S rRNA gene sequence as shown in FIG. 6 [SEQ ID NO:8].

### Processes of the Application

The present application also includes a process for the production of 1,3-propanediol (1,3-PD) comprising contacting a microorganism cultured from ethanol thin stillage with glycerol under conditions for the formation of 1,3-PD and, optionally, isolating the 1,3-PD. In a specific embodiment of the application the microorganism is Lactobacillus panis PM1A, Lactobacillus panis PM1B or Lactobacillus buchneri PM3. In a further specific embodiment, the microorganism is Lactobacillus panis PM1 deposited under accession number 180310-01 at the International Depository Authority of Canada located in, National Microbiology Laboratory, Public Health Agency of Canada, 1015 Arlington Street, Winnipeg, Manitoba, R3E 3R2, Canada on Mar. 18, 2010. In a further specific embodiment, the microorganism is Lactobacillus buchneri PM3 deposited under accession number 280910-01 at the International Depository Authority of Canada located in, National Microbiology Laboratory, Public Health Agency of Canada, 1015 Arlington Street, Winnipeg, Manitoba, R3E 3R2, Canada on Sep. 28, 2010. In a further embodiment of the application, the microorganism is further characterized as described above.

In an embodiment of the application the microorganism is a Lactobacillus microorganism that is cultured from thin stillage. In a further embodiment, the microorganism grows on agar or in broth at a pH of about 1 to about 6 and at temperatures of about 0° C. to about 45° C. In another embodiment, the agar is made by autoclaving thin stillage. In yet another embodiment, the microorganism is Lactobacillus panis PM1A or PM1B, or a mixture thereof, and possesses at least one, and suitably all, of the following DNA sequences: the DNA sequences for the 16S rRNA gene as shown in FIG. 1 [SEQ ID NO:1], the 16S-23S rRNA internal transcribed spacer (ITS) gene as shown in FIG. 2 [SEQ ID NO:2], the cpn60 gene as shown in FIG. 3 [SEQ ID NO:3], the glycerol dehydratase gene as shown in FIG. 4 [SEQ ID NO:4], [SEQ ID NO:5] and [SEQ ID NO:6] and the cobyric acid synthetase gene as shown in FIG. 5 [SEQ ID NO: 7]. In yet another embodiment, the microorganism is Lactobacillus buchneri PM3 and possesses the DNA sequences for the 16S rRNA gene as shown in FIG. 6 [SEQ ID NO:8].

In an embodiment the conditions for the formation of 1,3-PD comprise culturing the microorganism in the presence of a source of glycerol and other substances for the conversion of glycerol to 1,3-PD by the microorganism. In an embodiment of the application, the sources of glycerol are selected from by-products from biodiesel and ethanol production. In another embodiment the source of glycerol and other substances for the conversion of glycerol to 1,3-PD is thin stillage or distiller's solubles. In further embodiment, the source of glycerol is glycerol from a commercial source. In another embodiment the other substances for the conversion of glycerol to 1,3-PD comprise a source of food for the bacteria, for example, a source of glucose, xylose and/or sucrose. In a further embodiment the conditions for the conversion of glycerol to 1,3-PD comprise a reaction temperature of about 0° C. to about 45° C. The lower the temperature, the longer time is needed for conversion. For the example, the conditions for the conversion of glycerol to 1,3-PD may comprise a temperature of about 15° C. to about 25° C. and a time of about 5 days to about 9 days; a temperature of about 0° C. to about 10° C. and a time of about 3 weeks to about 5 weeks; or a temperature of about 25° C. to about 45° C. and a time of about 1 day to about 5 days. In a further embodiment the starting conditions for the conversion of glycerol to 1,3-PD comprise a reaction pH of about 3 to about 10.

The process for the conversion of glycerol to 1,3-PD using a microorganism of the present application may be performed as a batch or continuous process.

The isolation of 1,3-PD may be performed by known methods, such as, for example, distillation. Distillation of 1,3-propanediol may be accomplished by continuous or batch distillation of a concentrate produced after fermentation and evaporation of water. Alternatively separation of polyols like 1,3-PD from high molecular weight substances is possible through ultrafiltration and/or nanofiltration of the 1,3-PD containing fermentation broth. Such filtration would separate 1,3-PD as the membrane permeate and macromolecules as the retentate. 1,3-PD may be separated from ions present in the fermentation broth or a concentrate thereof by several methods known to those skilled in the art. For example, ion exclusion chromatography can be successfully used to separate salts present in the fermentation broth from 1,3-PD.

The present application also includes a process for the conversion of lactic acid to acetic acid comprising contacting a microorganism cultured from ethanol thin stillage with lactic acid under conditions for the formation of acetic acid and, optionally, isolating the acetic acid. In an embodiment the conversion of lactic acid to acetic acid is performed at the same time as the conversion of glycerol to 1,3-PD in a co-fermentation process. In a further embodiment, the two products, 1,3-PD and acetic acid, are each isolated using known procedures, for example, in batch or continuous distillation, extraction, filtration and/or chromatography.

### EXAMPLES

The following Examples are set forth to aid in the understanding of the invention, and are not intended and should not be construed to limit in any way the invention set forth in the claims which follow thereafter.

### Example I

Bacteria were isolated from thin stillage by dilution plating onto agar plates made of thin stillage and containing cycloheximide to inhibit the growth of most yeast and fungi. Due to the low pH of the stillage, agar was autoclaved separately and added after autoclaving to avoid hydrolyzing the agar. Bromophenol blue was added to these agar plates as a pH indicator to confirm that the thin stillage remained acidic after autoclaving and adding agar. Colonies with unique morphologies were selected and grown as pure strains, as well as inoculated into autoclaved thin stillage and incubated for 1 week. At the end of the 1 week incubation, the thin stillage was tested by ¹H NMR for conversion of glycerol to 1,3-PD. Cultures which were capable of converting the glycerol to 1,3-PD were subjected to DNA extraction and PCR for the 16S rRNA gene in order to determine the genus and species to which they belong. The efficacy of this method in screening for the diversity of organisms present was also tested by picking colonies directly from dilution plates and subjecting to DNA extraction and PCR of the 16S rRNA gene to determine the composition of the bacterial community on the dilution plates.

### Example 2

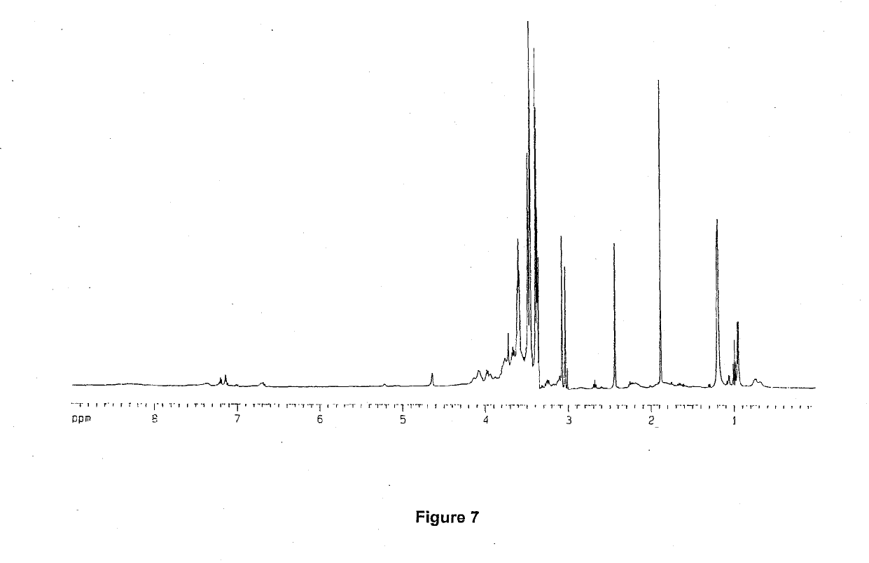

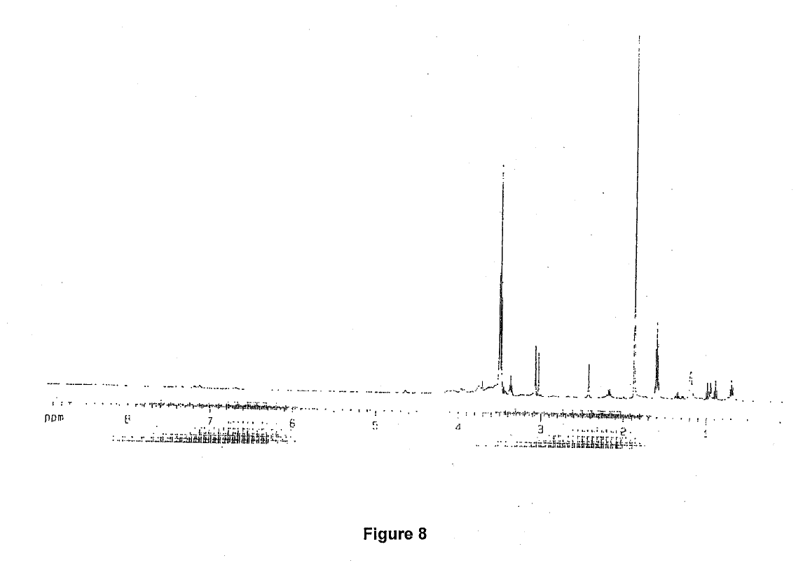

10 mL of autoclave sterilized thin stillage was inoculated with as little as 1 colony from an agar plate or 10 ul of broth culture containing the bacterial isolates. This thin stillage was incubated at a range of conditions, from 4° C. to 45° C. and conversion of the glycerol to 1,3-PD occurred in all instances. Conversion of glycerol to 1,3-PD was demonstrated by NMR (FIGS. 7 and 8). The ability of these organisms to convert glycerol to 1,3-PD was also tested at room temperature aerobically and anaerobically and conversion occurred. In control tubes of autoclaved thin stillage where no bacteria were added, the conversion did not occur in any instances. These experiments have been carried out at volumes ranging from 500 μl to 100 L. In all cases, the ‘converted’ stillage could subsequently be used to cause conversion of ‘natural’ stillage, whereas the non-innoculated stillage could not. Moreover, the bacteria isolates in question could be recovered from the converted stillage by agar plating and identified by DNA sequencing.

### Example 3

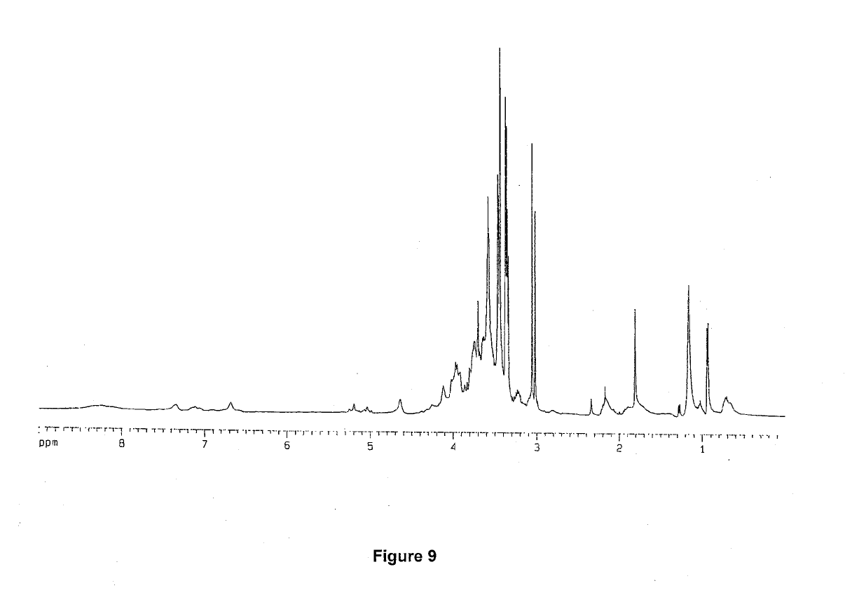

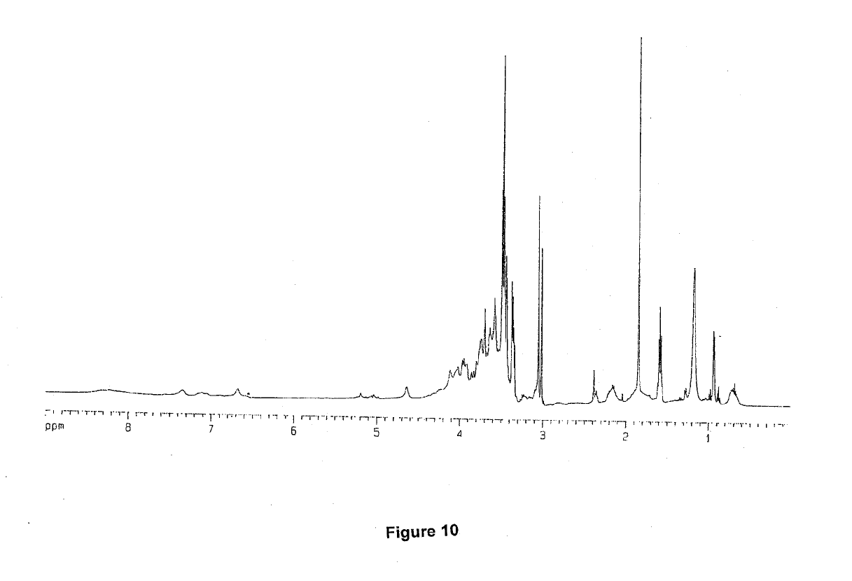

Using the same conditions reported in Example 2, distiller's solubles was diluted from 0%-75% v/v with water and sterilized by autoclaving. Results were the same as for Example 2 (i.e., conversion occurred only when the bacteria were added, see FIGS. 9 and 10). Non-autoclaved distiller's solubles were also tested with the same results.

### Example 4

Using the protocols from Examples 1 and 2, conditions were adjusted by using a temperature of 30° C. and with the addition of laboratory grade glycerol. Laboratory grade glycerol was added to thin stillage or diluted distiller's solubles at concentrations of 1, 2, 4, 6, 8, and 10% v/v. In all cases, conversion of glycerol to 1,3-PD occurred, although in some cases, the full amount of glycerol was not converted.

### Example 5

To confirm the homogeneity of cells in the bacterial innoculum, and their ability to convert the glycerol to 1,3-PD in thin stillage, 20 individual colonies were picked off an agar plate and used to inoculate respective tubes of autoclaved sterilized thin-stillage (a control tube with no bacteria was also included). The tubes were incubated for 1 week at 30° C. Each bacterial colony converted glycerol to 1,3-PD with an identical NMR profile. The control tube showed no conversion.

### Example 6

To test whether the bacteria experienced negative-feedback from buildup of 1,3-PD in solution, 1,3-PD was added to tubes containing non-converted thin stillage with bacteria added, as well as to control tubes. Laboratory grade 1,3-PD was added pre-incubation at concentrations of 1, 2, 4, 6, 8, and 10% v/v. No inhibition was observed from the presence of these concentrations of 1,3-PD, as demonstrated by the conversion of existing glycerol to additional 1,3-PD in all cases.

### Discussion

The bacteria isolated from thin stillage can be grown in laboratory de Mann Rogosa and Sharpe (MRS) broth or agar, or any ratio combination of sterilized thin stillage or distiller's solubles and MRS made into broth or agar. The bacteria grew with glucose, xylose, or sucrose as a sole carbon source, however, the bacteria did not grow on glycerol as a sole carbon source (i.e., they do not eat glycerin, they just convert it as part of their metabolism).

From this culture, inoculates containing from about 1000 to 1,000,000 bacteria (no upper or lower limits have been found) were transferred to tubes containing thin stillage, distiller's solubles (a concentrate of thin stillage made through evaporation, which was approximated 4× more concentrated than thin stillage), and any dilution of thin stillage or distiller's solubles with water. In all of these cases, the bacteria converted glycerin to 1,3-PD and lactic acid to acetic acid.

When the bacteria were transferred into tubes containing thin stillage or diluted distiller's solubles (<75% DS) and <20% (v/v) glycerol from biodiesel (this can be glycerol extracted at any point of the biodiesel production process and that optionally contains methanol), or laboratory grade glycerol, is added at the beginning of the fermentation, the glycerol is converted to 1,3-PD and lactic acid is converted to acetic acid. Adding a carbohydrate source (such as mash from a bioethanol fermentation) improved conversion of glycerol to 1,3-PD and lactic acid to acetic acid.

The above mentioned reactions occurred under sterile conditions, with only the bacteria added (i.e., sterilized by either autoclaving or gamma sterilization using a cobalt-60 gamma source) and under non-sterile conditions (i.e. thin stillage, distiller's solubles, glycerol and mash, used “as is”). Therefore, the bacteria of the present application are robust and can thrive and be active even when other microbes (e.g. bacteria, fungi and yeast) are in the fermentation solution. The bacteria were also active in thin stillage or distiller's solubles that originated from a fermentation which was treated with lactrol (aka Virginiamycin).

The activity of the bacteria was not highly dependent of steeping rate, as inoculations ranging from 1000 bacteria to 10,000,000 bacteria were tested and no significant variance was found.

The bacteria were shown to be active at temperatures ranging from 4° C. to 45° C. and the reaction proceeded with or without active shaking or stirring, in volumes ranging from 500 μl to greater than 100 L. Larger scale reactions are expected to be possible.

The bacteria were found to begin converting the glycerol to 1,3-PD and lactic acid to acetic acid in less than one day. Other compounds in thin stillage (or DS) were not affected, including α-glycerylphosphorylcholine (GPC), betaine, 2-phenethanol (PEA) and isopropanol.

The starting pH of the solution ranged from pH 3 to pH 10 and reaction occurred at all pH's tested. Regardless of starting pH, the bacteria decrease the pH of the solution to about 3.5-4.2.

Over 90% conversion of laboratory grade glycerol to 1,3-PD has been achieved in 48 hrs.

1. A microorganism cultured from ethanol thin stillage that converts glycerol and/or lactic acid into commercially useful products.

2. The microorganism of claim 1, wherein the microorganism belongs to the genus Lactobacillus.

3. The microorganism of claim 2, wherein the microorganism is Lactobacillus panis PM1A, Lactobacillus panis PM1B, or Lactobacillus buchneri PM3.

4. The microorganism of claim 1, wherein the microorganism is Lactobacillus panis PM1 deposited under accession number 180310-01 at the International Depository Authority of Canada located in, National Microbiology Laboratory, Public Health Agency of Canada, 1015 Arlington Street, Winnipeg, Manitoba, R3E 3R2, Canada on Mar. 18, 2010 or the microorganism is Lactobacillus buchneri PM3 deposited under accession number 280910-01 at the International Depository Authority of Canada located in, National Microbiology Laboratory, Public Health Agency of Canada, 1015 Arlington Street, Winnipeg, Manitoba, R3E 3R2, Canada on Sep. 28, 2010.

5. The microorganism of claim 1, wherein the microorganism possesses at least one of the following DNA sequences: 16S rRNA gene sequence as shown in FIG. 1 [SEQ ID NO:1]; 16S-23S rRNA internal transcribed spacer (ITS) gene sequence as shown in FIG. 2 [SEQ ID N0:2]; cpn60 gene sequence as shown in FIG. 3 [SEQ ID N0:3]; glycerol dehydratase gene sequence, as shown in FIG. 4 [SEQ ID N0:4], [SEQ ID N0:5] and [SEQ ID N0:6]; and cobyric acid synthetase gene sequence as shown in FIG. 5 [SEQ ID N0:7].

6. The microorganism of claim 1, wherein the microorganism possesses the following 16S rRNA gene sequence as shown in FIG. 6 [SEQ ID NO:8].

7. The microorganism of claim 1, wherein the commercially useful products are 1,3-propanediol and acetic acid.

8. A process for the production of 1,3-propanediol (1,3-PD) comprising contacting the microorganism of claim 1 with a source of glycerol under conditions for the formation of 1,3-PD and, optionally, isolating the 1,3-PD.

9. The process of claim 8, wherein the conditions for the formation of 1,3-PD comprise culturing the microorganism in the presence of a source of glycerol and other substances for the conversion of glycerol to 1,3-PD by the microorganism.

10. The process of claim 8, wherein the source of glycerol is by-products from biodiesel and ethanol production.

11. The process of claim 9, wherein the source of glycerol and other substances for the conversion of glycerol to 1,3-PD is thin stillage or distiller's solubles.

12. The process of claim 9, wherein the other substances for the conversion of glycerol to 1,3-PD by the microorganism comprise a food source for the microorganism.

13. The process of claim 8, wherein the isolation of 1,3-PD is by distillation.


SEQUENCE LISTING (8 sequences)

SEQ ID NO: 1 - DNA, 1434 bp, Lactobacillus panis
tgcaagtcgagcgcactggcccaactgatatgacgtgcttgcattgatttgacgatggattaccagtgagcggcggacgggtgagtaacacgtgggcaacctgccctaaagcgggggataacatttggaaacaggtgctaataccgcataactacgaaaaccacatggttttcgtatcaaagatggtttcggctatcactttaggatgggcccgcggtgcattagctagttggtagggtaacggcctaccaaggcaatgatgcatagccgagttgagagactgatcggccacaatggaactgagacacggtccatactcctacgggaggcagcagtagggaatcttccacaatgggcgcaagcctgatggagcaacaccgcgtgagtgaagaagggtttcggctcgtaaaactctgttgttgaagaagaacgtgcatgagagtaactgttcatgcagtgacggtattcaaccagaaagtcacggctaactacgtgccagcagccgcggtaatacgtaggtggcaagcgttatccggatttattgggcgtaaagcgagcgcaggcggttgcttaggtctgatgtgaaagccttcggcttaaccgaagaagtgcatcggaaaccgggcgacttgagtgcagaagaggacagtggaactccatgtgtagcggtggaatgcgtagatatatggaagaacaccagtggcgaaggcggctgtctggtctgcaactgacgctgaggctcgaaagcatgggtagcgaacaggattagataccctggtagtccatgccgtaaacgatgagtgctaggtgttggagggtttccgcccttcagtgccgcagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgctaacctaagagattaggcgttcccttcggggacgcaatgacaggtggtgcatggtcgtcgtcagctcgtgtcgtgagatgttgggttaagtcccgcaacgagcgcaacccttgttactagttgccagcattcagttgggcactctagtgagactgccggtgacaaaccggaggaaggtggggacgacgtcagatcatcatgccccttatgacctgggctacacacgtgctacaatggccggtacaacgagcagctaacccgcgagggtgtgcaaatctcttaaagccggtctcagttcggactgcagtctgcaactcgactgcacgaagtcggaatcgctagtaatcgcggatcagcatgccgcggtgaatacgttcccgggccttgtacacaccgcccgtcacaccatggaagtttgcaatgcccaaagtcagtggcctaaccatttgga

SEQ ID NO: 2 - DNA, 522 bp, Lactobacillus panis
gtcagtggcctaaccattttggagggagctgcctaaggcagggcagatgactggggtgaagtcgtaacaaggtagccgtaggagaacctgcggctggatcacctcctttctaaggaataaaacggaacctacacattagttgaaactttgtttagttttgaggggtttaccctcagagtttgtactttgaaaactaaataatatctaatttctttattaatcaaaacaataaaccgagaacaccgcgttatttgagttttattaacgaaataatcgctaactcaattaatcaaggtgatcacgaagtgatcatcaaggttaagttatgaagggcgcatggtgaatgccttggtactaggagccgatgaaggacgggataaacaccgatatgcttcggggagtggtaagtacactttgatccggagatttccgaatggggaaacccaaccaacttagtcgttggttatctgactagtgaattcatagctagcagagggaagacgcagtgaactgaaacatcta

SEQ ID NO: 3 - DNA, 621 bp, Lactobacillus panis
gtcacgacgagggggcgggggatgggacgacgactgctaccgttttaacccaagcaattgtcaatgccggaatgaagaacgttacttctggtgctaacccagttggtattcgtcgcgggattgaaaaggctactgcagttgcagttgatgcattaaagaagatgtcacacgacgttaagaccaagagtgacattgaacaaatcgcttctatttcagctgctaaccctgaagttggtaagttaattgctgacgccatggaaaaggttggccacgatggtgttatcaccattgaagattctcgtggtgttgacacgagtgttaacgtggttgaagggatgagctttgaccgtggttacatgtctcaatacatggtaactgacaatgataagatggaagctgacttggataacccatacatcctgattactgacaagaagatcagcaatattcaagacatcttgccattactgcaaaacgttgttcagcaaggccgggctctgttgatcattgccgatgacattaccggtgaagctctgccaacccttgttttgaacaagattcgtggtaccttcaacgtatgtgccgttaaggcccccggcttcggcgacaacagtcctgtgt

SEQ ID NO: 4 - DNA, 1677 bp, Lactobacillus panis
atgaaacgtcaaaaacgatttgaagcattgaaaaagcggccgattcataaagatacattcgttgatgaatggccagatgaaggatttgttgcaatgatgggtcctaatgaccctaagccaagtatcaaagttgaaaatggtgtcatcactgaaatggatggcaagaagcgtgaagactttgatttgattgacttatacattgcaaagtacgccatcaatattgacaacgttgaaaaggttatggcaactgattcaaccaagattgcacggatgcttgttgatccgaacgtatcacgggatgaagttttgaagtacacttctgctttaaccccagctaaagccgaagaagttatcagtaagcttgacttcggtgaaatgatcatgggtgttaagaagatgcgtccacggtacaagccagctaaccagtgtcacgttaccaacactgttgataacccagttgaaattgctgctgatgctgccgaagctgctcttcgtggcttcccagagcaagaaactactactgcggttgctcgttacgcaccgttcaacgccatttcgattctggttggttcacaaactggacgtccaggtgttcttacccaatgttcagtcgaagaagctactgaacttcaactaggaatgcgtgggttcactgcttatgccgaaaccatttctgtttatggtactgaccgtgtatttaccgatggtgacgatactccatggtctaaaggattcttggcatcatgctatgcatcacgtggtttgaagatgcggtttacttcaggtgccggttcagaagtcttgatgggttacccagaaggtaagtcaatgctttaccttgaagctcgttgtatcttacttaccaagggttccggtgttcaaggcctgcaaaacggtgctgtaagttgtatttcgatgccgggtgccgttcctaatggtttacgtgaagtccttggtgaaaaccttctttgcatgatgtgcgatatcgaatgtgcttctggttgtgaccagacattctcagcatcagacatgcggcggactgaacggtttatgggtcaattcatcggtggtactgactacatttgttctggatacgctgccgaagataactatgataacacctttgccggatcaaacaccgatgtcaatgactatgatgatatgtacgtcatggaacgtgaccttggtcaaaactatggtattcacccagtaagtgaagaagatgttattaagatccggaacaaggctgctaaggctcttcaagctgtctttgatgcccttggccttcctaagattactgatgaagaagttgaagctgctacctatgcaaatactcatgatgatatgccaaagcgtgacatggttgcagatatgaaggctgctcaaaacatgatggaccgtggcattaccgcgattgaccttatcaaggcattagctgataatggttatcctgaagttgctcaagctatccttgaccttcaaaagcaaaaggtttgcggtgactaccttcaaacttcatcaatctttgatagcaagtggaacgttaactcagctgtcaacaatccaaacacttatcaaggaccaggtacaggttatcgtctatatgaagataaagatgaatggaagaagattaaggctcttccatgggctcttgatcctgaacatctaaaactgtaa

SEQ ID NO: 5 - DNA, 699 bp, Lactobacillus panis
atggctgatattgatgagaacttagtacgacagatagtacaagaagttctagcacaaacaaagaatgtcgatacaccaattgattttggtaaaaatagttctacagcaactgccactaagcaacaagctaacagtaaagcagtccctgaaaagaaagttgactggttccaaccagtaggtgaggctaagccaggttactcgaaggatgaagttgtaattgctgttgggccagcatttgcaactgttcttgataaaacgatgactggtgttccccacaaggaagttcttcgtcaggtaattgccggaattgaagaagaagggcttaaggcacgggttgttaaagtctaccggacttcagatgttggtttcatggctgtccaaggtgatcacctatctggatcaggaattgctgtcggtatccaatccaagggtacggcaatcattcaccaaaaggatgaagacccactgagtaacttggaattgttcccacaagctccagttttgactccagaaacattccgggcaattggtaagaatgcggcaatgtacgctaagggcgagactcctgaaccagttccagcagttaacgatgccttagcacgtgctcactaccaagcaattgctgccattatgcacattcgggaaacccaccaagtagttgttggtaagcctgaagaagaaattaaggtaacattttaa

SEQ ID NO: 6 - DNA, 516 bp, Lactobacillus panis
atgagcgaaattgacgatttagttaaaaagataactcagcaacttggagagcaatccaccagtgctgctagttcaaagacagggaccacttctgttccggataacatgggcagaaatgattacccactttatggcaagcaccgtaacctggtaaaaacaccaacaggcaagaaccttgacgacattaatctaaatagtattaagaatgaccaaatcaagggtgacgaaatgcggattacccctgaggcattgaagatgcaaggtgaaattgctgcttcagctggtcgtcctgctattcaaaagaacttccaacgggctgctgaattaactaaggttccagatgctcggttactgcaaatgtacaatgctttacggccataccgttccagcaagcaagatcttcttgatattgctgatgaactccgtaacaagtacaacgctccaatctgtgctgcttggtttgaagaagccgctaagtactacgaaagccggaagaagcttaagggcgacaactag

SEQ ID NO: 7 - DNA, 1509 bp, Lactobacillus panis
atggtagtgcaatcagtaatgtttcaaggaaccgcatcggattctggaaaaagctggatggctgcagcggtttgccgacttttgacaaatcaagggaaaaaggttgcaccgtttaaatcacaaaacatggccctaaactcgtttatcacggatcaaggtgatgagatggggcgggcacaggtttaccaggcggaggcagcccgggtgaaaccagacgttagaatgaatccaatcctgctaaaaccctcaactgatcaagattcgcaagtgatagtaatggggaaagtactagccgatatggacgcagctagttattataagtttaagcctcaactaattccagatataaagaaagcttaccaaggacttgccattgagaatgatgccattattttagagggagcaggaagcccagcggagattaacttaaatgaaaatgatattgtaaatatgggaatggcgcgaatggctgctgcgccggtgatccttgttgccgatatcgacaagggaggtgtctttgcatccatttatggcaccatcaaattactgacaaaggaagaccagcaacgaatcaagggaattattattaacaagtttcgtggtgataagacgctcttggagccggggaacaagatgattgaaaagctgaccggtgttcctgttattggggtgttaccgatgagtgacgtcgatattgatgaggaagatagtgtttcgctagtaaggaagccgcggcaaaagaacccggctaaggcccttgatgttgcggttgttgaccttaacaaaatctcgaattttactgatatccatagtctaaaaattcagccggacgtgtctgttcgctacgttctcaaggcagaagaacttggtaccccagacttattaatcctccctggtagtaaaaatacaaacgaggatctcgcctctttgaaaagaaacggtttggctaaagcgattatacatgcccatgctgacgggagtgtagtgatcggcatttgcggtggttaccaaattctgggtcggacgcttcgtgatcccaatggaattgaatcaccaatcaaggagcagacagggctgggcctccttgatacagaaaccattttcaacgagcgaaaaactacaacccaggcaaccgctaaacgcaagaattatgttcttaagggctatgaaatccatatgggagcaacgaaactcggtccgaatacaacaccattttcaattattcaagaaactaatggcgaagcagagcagcgggaagacggggcagttaacgctgacgaaacagttatcggaacttaccttcacggaatctttgataatccttattggacacgccacctcttaaatcggcttcgggttgctaaggggttagcaccattagttaataccatggtttctatcagtgagtataaagatcaacagtatgaaaaacttgcgaaactatttgaagaaaatgttgatatgaagaagtttagtcagattctacaggattcaacaagggatgattaa

SEQ ID NO: 8 - DNA, 499 bp, Lactobacillus buchneri
cgcgtctccgttgatgattttaggtgcttgcacttgaaagatttaacattgagacgagtggcgaactggtgagtaacacgtgggtaacctgcccttgaagtaggggataacacttggaaacaggtgctaataccgtataacaaccaaaaccacctggttttggtttaaaagacggcttcggctgtcactttaggatggacccgcggcgtattagcttgttggtaaggtaacggcctaccaaggcgatgatacgtagccgacctgagagggtaatcggccacattgggactgagacacggcccaaactcctacgggaggcagcagtagggaatcttccacaatggacgaaagtctgatggagcaacgccgcgtgagtgatgaagggtttcggctcgtaaaactctgttgttggagaagaacaggtgtcagagtaactgttgacatcttgacggtatccaaccagaaagccacggctaactacgtgccagcagccggcggt